(19) United States
(12) Patent Application Publication
(10) Pub. No.: US 2024/0260653 A1
(43) Pub. Date: Aug. 8, 2024

(54) INHALER

(71) Applicant: JEZEL INC., MONSEY, NY (US)

(72) Inventors: Sharon SHINEBERG, RAMAT GAN (IL); Oren Manoach KARPAS, YESUD HAMA'ALA (IL); Moshe ELIYAHU, NEHUSHA (IL)

(21) Appl. No.: 18/429,500

(22) Filed: Feb. 1, 2024

(30) Foreign Application Priority Data
Feb. 6, 2023 (IL) 300452

(51) Int. Cl. A24F 40/10; A24F 40/485; A24F 40/50

(52) U.S. Cl. CPC A24F 40/10; A24F 40/485; A24F 40/50

(57) ABSTRACT

An inhaler including a first chamber including a mouthpiece for inhaling thereby, at least two inlets, and a controllable valve for each of the at least two inlets, a controller for controlling the controllable valves, and a cartridge for being attachable to each of the at least two inlets, each of the cartridges for containing a different substance, thereby the controller controls inhalation ratio between the substances.

### CROSS-REFERENCE TO RELATED APPLICATIONS

This application claims the benefit of priority from Israel Patent Application No. 300452, filed Feb. 6, 2023, the disclosure of which is incorporated herein by reference.

### TECHNICAL FIELD

The invention relates to the field of inhalers.

### BACKGROUND

For the needs of smoking the cannabis plant, there are several operations of smoking, for example a vaporizer. The existing viper produces vaporization of a uniform extraction of all the terpenes and the chemicals are mixed together and this causes the user to lose some of the unique properties of the terpenes.

There is a long felt need to provide a solution to the above-mentioned and other problems of the prior art.

### SUMMARY

An inhaler, including:
a chamber including controllable valves;
cartridges for containing different substances,

thereby controlling inhalation ratio between the substances.

### BRIEF DESCRIPTION OF THE DRAWINGS

Embodiments, features, and aspects of the invention are described herein in conjunction with the following drawings:

FIG. 1 depicts an apparatus for producing at least two substances.

FIG. 2 depicts an inhaler according to one embodiment of the invention.

FIG. 3 depicts a storage device of FIG. 2.

FIG. 4 depicts the inhaler of FIG. 2, using a different dosage ratio.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

The invention, in embodiments thereof, relates to a variable dose wafer containing a method of isolation and separation between the terpenes and cannabinoids.

The current viper contains cartridges containing liquids of unique extracts of terpenes and cannabinoids that are differentiated and isolated from each other.

The viper contains one or more central cartridges containing a heating element and liquids for example CBD, THC, etc.

The heating element creates vaporization of the liquids found in the above-mentioned cartridge at a selected temperature, for example 270 degrees Celsius. These vapors pass through a medium, for example a tube, to the user.

These vapors pass through a medium containing, for example, combinatory nozzles with a selected flow resistance of liquids

The nozzles/valves are conductive and connected to the aforementioned cartridges during the flow of the vapors in between the nozzles are opened, for example, mechanically, electrically, etc. and release in a combinatorial way selected doses of the desired terpenes from the cartridges.

The flow of liquids from the cartridges can be applied by utilizing the principle of a venturi neck, for example the above method will allow the user to consume chemicals in a selected order which will cause the user selected phenomena which will cause him, for example, to go from a feeling of mental stress to a feeling of mental calm and vice versa or a change from a feeling of pain to a feeling of relief in pain etc.

Each of the valves of the nozzles is controllable, for example, in an electro-mechanical, electrical-mechanical way, etc., which will allow in real time to create combinatory doses according to the user's or doctor's choice.

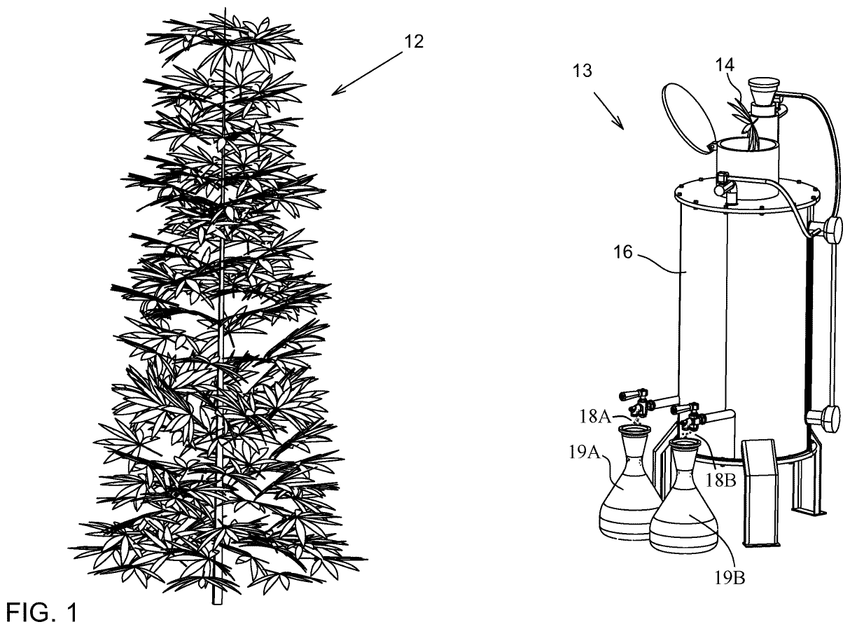

FIG. 1 depicts an apparatus for producing at least two substances.

A portion 14 of cannabis plant 12 is inserted into apparatus 13, which produces substances 18A and 18B, typically being liquids, from portion 14, into compartments 19A and 19B respectively. Substance 18A may be Tetrahydrocannabinol (THC) whereas substance 18B may be Cannabidiol (CBD).

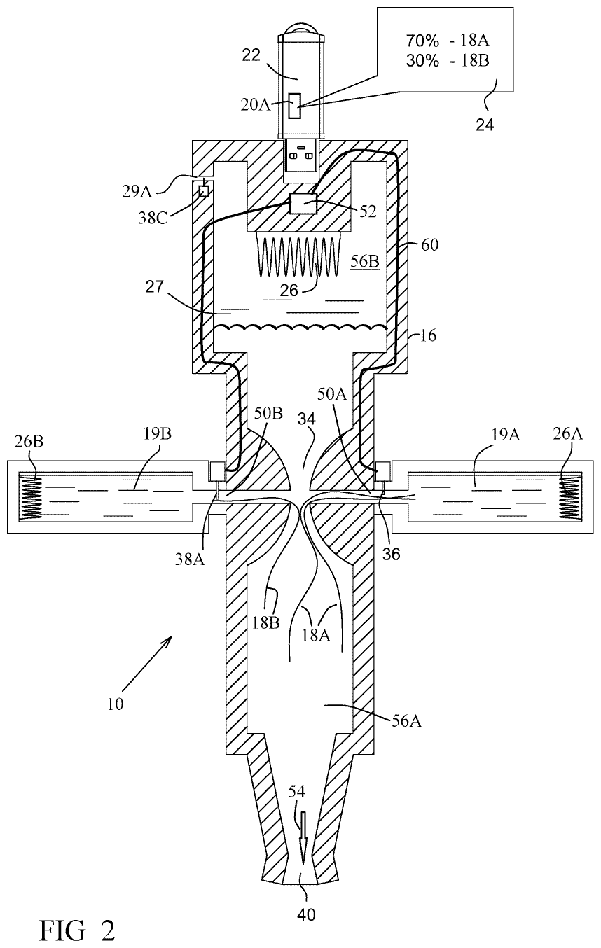

FIG. 2 depicts an inhaler according to one embodiment of the invention.

An inhaler 10 according to one embodiment of the invention, includes a mouthpiece 40; a chamber 56A ending with mouthpiece 40; a removable cartridge 19A containing substance 18A; an inlet 50A including a controllable valve 38A, for controlling intake of substance 18A from cartridge 19A; a removable cartridge 19B containing substance 18B from cartridge 19B; an inlet 50B including a controllable valve 38B, for controlling intake of substance 18B; and a controller 52 for controlling valves 38A and 38B.

Thus, inhaling 54 by mouthpiece 40 mixes substances 18A and 18B within chamber 56A. The ratio between substances 18A and 18B is determined by the ratio of the opening extent valves 38A and 38B.

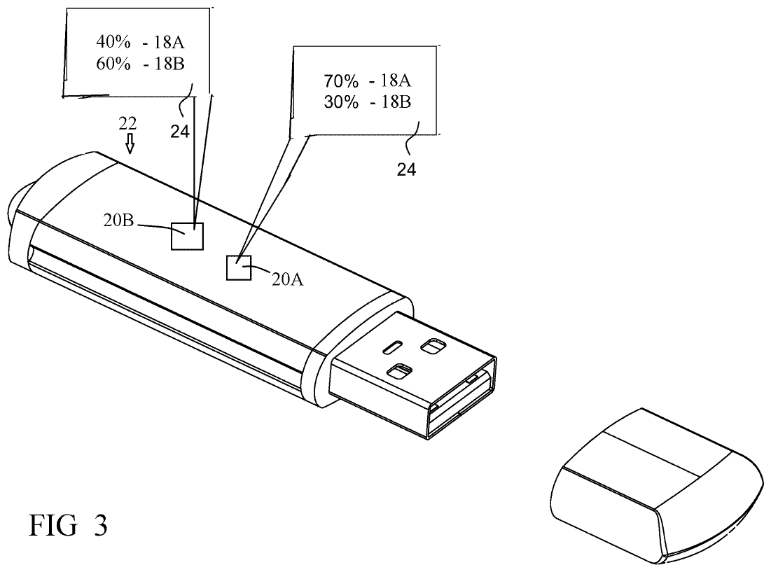

FIG. 3 depicts a storage device of FIG. 2.

Inhaler 10 preferably may be controllable by a pluggable storage device 22, e.g. flash drive USB, including files 20A, 20B, etc.

The content/data 24 of file 20A may be the ratio of 70% of substance 18A and 30% of substance 18B (together 100%), which may be effective for treating a first illness; and the content of file 20B may be the ratio of 40% of substance 18A and 60% of substance 18B, which may be effective for treating a second illness.

Referring again to FIG. 2, upon selecting file 20A having ratio of 70% of substance 18A and 30% of substance 18B, controller 22 opens valve 38A more than valve 38B as shown in FIG. 2, thus intaking substance 18A more than substance 18B as shown in FIG. 2.

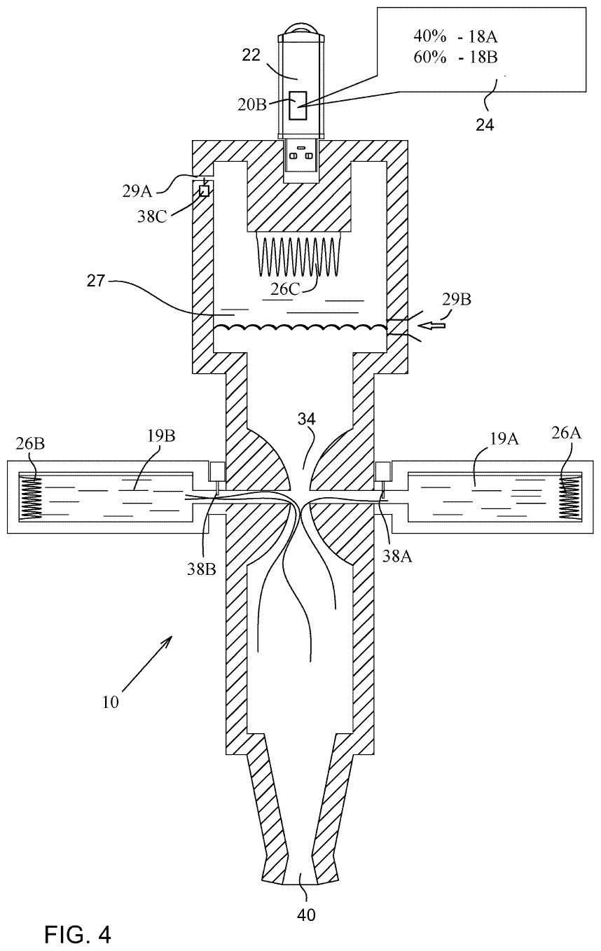

FIG. 4 depicts the inhaler of FIG. 2, using a different dosage ratio.

Upon selecting file 20A having ratio of 40% of substance 18A and 60% of substance 18B, controller 22 opens valve 38A less than valve 38B as shown in FIG. 4, thus intaking substance 18A less than substance 18B as shown in FIG. 4.

Rather or in addition to the control of the consuming ratio by controlling the extent of opening valves 38A and 38B, controller 52 may increase the amount of releasing substance 19A by heating a heater 26A for heating substance 19A.

The Venturi effect is the reduction in fluid pressure that results when a fluid flows through a constricted section, named herein “Venturi neck”.

Inhaler 10 further includes a chamber 56B; and a venturi neck 34 disposed between chambers 56A and 56B, for intaking substances 19A and 19B.

Chamber 56B may include an air inlet 29A having a valve 38C, being controllable by controller 52 reading from file 20A or 20B; and a heater 26C for heating the content of chamber 56B.

Chamber 56B may further include an inlet 29B for inserting a dissolvent 27.

Thus, in one aspect, the invention is directed to an inhaler (10), including:
a first chamber (56A) including:
   a) a mouthpiece (40) for inhaling (54) thereby;
   b) at least two inlets (50A,50B); and
   c) a controllable valve (38A,38B) for each of the at least two inlets (50A,50B);
a controller (52) for controlling the controllable valves (38A,38B); and
a cartridge (19A,19B) for being attachable to each of the at least two inlets (50A,50B), each of the cartridges (19A,19B) for containing a different substance (18A,18B),

thereby the controller (52) controls inhalation ratio between the substances (18A,18B).

The inhaler (10) may further include:
a storage device (22), for being pluggable to the inhaler (10), for including data (24) including the inhalation ratio, for being readable by the controller (22).

The inhaler (10) may further include:
a second chamber (56B); and
a Venturi neck (34) disposed between the first (56A) and second (56B) chambers,

wherein the at least two inlets (50A,50B) communicate with the Venturi neck (34).

The inhaler (10) may further include:
a heating element (26A,26B) for each of the at least two inlets (50A,50B); being controllable by the controller (52), for further controlling the inhalation ratio between the substances (18A,18B).

The inhaler (10) may further include:
an air inlet (29A) including a third valve (38C) being controllable by the controller (52).

The inhaler (10) may further include:
an inlet (29B) for administering a dissolvent (27).

In the figures and description herein, the following reference numbers (Reference Signs List) have been mentioned:
number 10 denotes the inhaler according to one embodiment of the invention;
12: cannabis plant or other raw material;
13: apparatus for producing substances 18A,18B;
14: portion of cannabis plant 12;
16: tube;
18A,18B: substances for being inhaled;
19A,19B: compartments, which may be cartridges;
20A,20B: files containing data 24;
22: storage device;
24: data including mixing ratio of substances 18A,18B, etc.;
26A,26B,26C: heating elements controlled by controller 22;
27: dissolvent or other substance being inhaled;
29A: air inlet;
29B: dissolvent inlet;
34: Venturi neck;
38A,38B: valves controlled by controller 22;
40: mouthpiece;
50A,50B: inlets;
54: inhaling;
56A,56B: chambers within tube 16;
60: electric wires for controlling valves 38A,38B;

The foregoing description and illustrations of the embodiments of the invention have been presented for the purpose of illustration, and are not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

What is claimed is:

1. An inhaler, comprising:
a first chamber comprising:
a) a mouthpiece for inhaling thereby;
b) at least two inlets; and
c) a controllable valve for each of said at least two inlets;
a controller for controlling said controllable valves; and
a cartridge for being attachable to each of said at least two inlets, each of said cartridges for containing a different substance,
thereby said controller controls inhalation ratio between said substances.

2. The inhaler according to claim 1, further comprising:
a storage device, for being pluggable to said inhaler, for comprising data comprising said inhalation ratio, for being readable by said controller.

3. The inhaler according to claim 1, further comprising:
a second chamber; and
a Venturi neck disposed between said first and second chambers,
wherein said at least two inlets communicate with said Venturi neck.

4. The inhaler according to claim 1, further comprising:
a heating element for each of said at least two inlets; being controllable by said controller, for further controlling said inhalation ratio between said substances.

5. The inhaler according to claim 1, further comprising:
an air inlet comprising a third valve being controllable by said controller.

6. The inhaler according to claim 1, further comprising:
an inlet for administering a dissolvent.
